(19) United States
(12) Patent Application Publication
(10) Pub. No.: US 2023/0210501 A1
(43) Pub. Date: Jul. 6, 2023

(54) ULTRASOUND CONTRAST IMAGING METHOD AND DEVICE AND STORAGE MEDIUM

(71) Applicant: SHENZHEN MINDRAY BIO-MEDICAL ELECTRONICS CO., LTD., Shenzhen (CN)

(72) Inventors: Aijun WANG, SHENZHEN (CN); Muqing LIN, SHENZHEN (CN); Yaoxian ZOU, SHENZHEN (CN); Maodong SANG, SHENZHEN (CN); Xujin HE, SHENZHEN (CN)

(21) Appl. No.: 18/081,300

(22) Filed: Dec. 14, 2022

Related U.S. Application Data
(63) Continuation of application No. PCT/CN2020/096627, filed on Jun. 17, 2020.

(51) Int. Cl. A61B 8/08; A61B 8/00

(52) U.S. Cl. CPC A61B 8/483; A61B 8/466; A61B 8/5238

(57) ABSTRACT

Provided are a CEUS imaging method, an ultrasound imaging apparatus and a storage medium. The method includes: controlling an ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data; rendering a second contrast data and a second tissue data in real time to acquire a hybrid rendered image of the second contrast data and the second tissue data, the second contrast data containing all or part data of the first contrast data, and the second tissue data containing all or part data of the first tissue data; and displaying the hybrid rendered image in real time. The CEUS imaging method and the ultrasound imaging apparatus according to embodiments of the present disclosure help users more intuitively understand and observe the real-time spatial position relationship of a contrast agent in tissues, and further acquire more clinical information.

### TECHNICAL FIELD

The present disclosure relates to ultrasound imaging, and more specifically to contrast enhanced ultrasound (CEUS) imaging methods, ultrasound imaging apparatus and storage media.

### BACKGROUND OF THE INVENTION

Ultrasonic instruments are generally used by doctors to observe the internal tissue structures of a human body. Doctors can get ultrasonic images of the human body by placing an operating probe onto a skin surface corresponding to a body part. Ultrasound has become a main auxiliary means for doctors to diagnose because of its safety, convenience, nondestructive, cheap and other characteristics.

Ultrasound contrast agents, a substance used to enhance image contrast in ultrasound imaging, are generally encapsulated micro-bubbles with a diameter of microns. The micro-bubbles having strong acoustic impedance are entered into a blood circulation system through intravenous injection to enhance ultrasonic reflection intensity to achieve CEUS imaging, significantly improving the detection of diseased tissues in micro-circulation perfusion, compared with conventional ultrasound imaging. Ultrasound contrast agents have become a very important technological means in ultrasonic diagnosis due to its advantages of simplicity, short time consumption, real time, non-invasion and non-radiation, compared with other examination methods such as computed tomography (CT), magnetic resonance imaging (MRI).

3D contrast imaging refers to a series of computer processing of dynamic 2D section contrast data continuously collected which are then rearranged to form 3D data in accordance with a certain order and then restored 3D structure information about tissues and organs by using 3D rendering technology (surface rendering, volume rendering, etc.), helping doctors make more detailed clinical diagnosis. Medical 3D CEUS imaging technology has been widely used in examination of thyroid (nodule detection), breast, liver (sclerosis, nodule, tumor), oviduct (obstructed) and so on.

Only 3D contrast enhanced images or only tissue images are displayed separately in most 3D CEUS imaging at present. However, the image information and relative spatial position relationship of both the two kinds of images may need to be combined so as to accurately locate and diagnose related lesions; to this end, there may need to switch between the 3D contrast enhanced images and the tissue images repeatedly by users, leading to complicated operations and a certain amount of spatial imagination to determine the spatial position relationship therebetween.

### SUMMARY OF THE INVENTION

A CEUS imaging scheme that enables users to more intuitively understand and observe the spatial position relationship of contrast agent in tissues so as to obtain more clinical information is provided in the present disclosure. The CEUS imaging scheme proposed herein is briefly illustrated below, and more details thereof will be described in the following Detailed Description in conjunction with attached drawings.

A contrast enhanced ultrasound imaging method provided in accordance with an aspect of the present disclosure may include: controlling an ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data; rendering a second contrast data and a second tissue data in real time to acquire a hybrid rendered image of the second contrast data and the second tissue data, the second contrast data containing all or part data of the first contrast data, and the second tissue data containing all or part data of the first tissue data; and displaying the hybrid rendered image in real time.

An ultrasound imaging apparatus provided in accordance with another aspect of the present disclosure may include an ultrasonic probe, a transmitting/receiving sequence controller, a processor and a display, the transmitting/receiving sequence controller configured for controlling the ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data; the processor configured for rendering a second contrast data and a second tissue data in real time to acquire a hybrid rendered image of the second contrast data and the second tissue data, the second contrast data containing all or part data of the first contrast data, and the second tissue data containing all or part data of the first tissue data; and the display configured for displaying the hybrid rendered image in real time.

A storage medium provided in accordance with yet another aspect of the present disclosure may store thereon a computer program which, when being executed, may implement the contrast enhanced ultrasound imaging method mentioned above.

With the CEUS imaging methods, the ultrasound imaging apparatus and the storage media according to embodiments of the present disclosure, volumetric contrast data and volumetric tissue data are collected simultaneously and then fused and rendered to acquire a hybrid rendered image, which can help users more intuitively understand and observe the real-time spatial position relationship of a contrast agent in tissues, and further acquire more clinical information.

### BRIEF DESCRIPTION OF THE DRAWINGS

FIG. 1 is a schematic block diagram of an exemplary ultrasound imaging apparatus used to implement a CEUS imaging method according to an embodiment of the present disclosure;

FIG. 2 is a schematic flowchart of a CEUS imaging method according to an embodiment of the present disclosure;

FIG. 3 is a schematic flowchart of acquiring volumetric contrast data and volumetric tissue data in a CEUS imaging method according to an embodiment of the present disclosure;

FIG. 4 is a schematic flowchart of an example of fusing and rendering volumetric contrast data and volumetric tissue data in a CEUS imaging method according to an embodiment of the present disclosure;

FIG. 5 is a schematic flowchart of another example of fusing and rendering volumetric contrast data and volumetric tissue data in a CEUS imaging method according to an embodiment of the present disclosure;

FIG. 6 is an exemplary schematic diagram of a hybrid rendered image acquired by a CEUS imaging method according to an embodiment of the present disclosure;

FIG. 7 is a schematic block diagram of an ultrasound imaging apparatus according to an embodiment of the present disclosure; and

FIG. 8 is a schematic block diagram of an ultrasound imaging apparatus according to another embodiment of the present disclosure.

### DETAILED DESCRIPTION

In order to make the objectives, technical solutions, and advantages of the present disclosure clearer, example embodiments according to the present disclosure will be described in detail below with reference to the accompanying drawings. Apparently, the described embodiments are merely some rather than all of the embodiments of the present disclosure. It should be understood that the example embodiments described herein do not constitute any limitation to the present disclosure. All other embodiments derived by those skilled in the art without creative efforts on the basis of the embodiments of the present disclosure described herein shall fall within the scope of protection of the present disclosure.

In the following description, a large number of specific details are given to provide a more thorough understanding of the present disclosure. However, it would be understood by those skilled in the art that the present disclosure can be implemented without one or more of these details. In other examples, to avoid confusion with the present disclosure, some technical features known in the art are not described.

It should be understood that the present disclosure can be implemented in different forms and should not be construed as being limited to the embodiments presented herein. On the contrary, these embodiments are provided to make the disclosure thorough and complete, and to fully convey the scope of the present disclosure to those skilled in the art.

The terms used herein are intended only to describe specific embodiments and do not constitute a limitation to the present disclosure. When used herein, the singular forms of “a”, “an”, and “said/the” are also intended to include plural forms, unless the context clearly indicates otherwise. It should also be appreciated that the terms “comprise” and/or “include”, when used in the specification, determine the existence of described features, integers, steps, operations, elements, and/or units, but do not exclude the existence or addition of one or more other features, integers, steps, operations, elements, units, and/or groups thereof. As used herein, the term “and/or” includes any and all combinations of relevant listed items.

For a thorough understanding of the present disclosure, detailed steps and detailed structures will be provided in the following description to explain the technical solutions proposed by the present disclosure. The preferred embodiments of the present disclosure are described in detail as follows. However, in addition to these detailed descriptions, the present disclosure may further have other implementations.

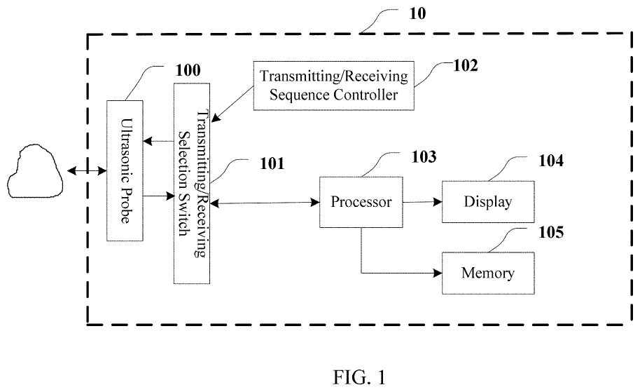

First, an exemplary ultrasound imaging apparatus for realizing a CEUS imaging method according to an embodiment of the present disclosure will be described with reference to FIG. 1.

FIG. 1 shows a schematic block diagram of an exemplary ultrasound imaging apparatus 10 used to implement a CEUS imaging method according to an embodiment of the present disclosure. As shown in FIG. 1, the ultrasound imaging apparatus 10 may include an ultrasonic probe 100, a transmitting/receiving selection switch 101, a transmitting/receiving sequence controller 102, a processor 103, a display 104 and a memory 105. The transmitting/receiving sequence controller 102 may excite the ultrasonic probe 100 to transmit ultrasonic waves to a target object (a test object), and may also control the ultrasonic probe 100 to receive ultrasonic echoes from the target object to acquire ultrasonic echo signals/data. The processor 103 may process the ultrasonic echo signals/data to acquire tissue-related parameter(s) and ultrasonic image(s) of the target object. The ultrasonic image acquired by the processor 103 may be stored in the memory 105 and be displayed in the display 104.

In the embodiment of the present disclosure, the display 104 of the ultrasound imaging apparatus 10 mentioned above may be a touch screen, a liquid crystal display screen, etc., or an independent display device (such as a liquid crystal display, a television set, etc.) independent of the ultrasound imaging apparatus 10, or a display screen on a mobile phone, tablet computer and other electronic devices.

In the embodiment of the present disclosure, the memory 105 of the ultrasound imaging apparatus 10 mentioned above may be flash memory card, solid-state memory, hard disk, etc.

A computer-readable storage medium may be also provided in an embodiment of the present disclosure. The computer-readable storage medium may store a plurality of program instructions which may be called and executed by the processor 103 to execute some or all steps or any combination of the steps in the CEUS imaging method according to embodiments of the present disclosure.

In one embodiment, the computer-readable storage medium may be the memory 105, which may be flash memory card, solid-state memory, hard disk and other non-volatile storage media.

In an embodiment of the present disclosure, the processor 103 of the ultrasound imaging apparatus 10 mentioned above may be implemented by software, hardware, firmware or a combination thereof, and may use circuits, single or multiple application specific integrated circuits (ASICs), single or multiple general integrated circuits, single or multiple microprocessors, single or multiple programmable logic devices, or a combination of the above circuits or devices or other suitable circuits or devices, so that the processor 103 can execute corresponding step(s) of the CEUS imaging method in various embodiments.

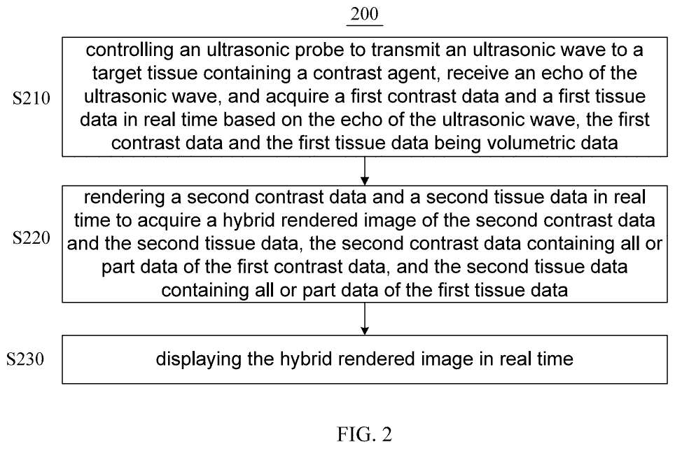

The CEUS imaging method according to the present disclosure, which may be executed by the ultrasound imaging apparatus 10 mentioned above, may be described in detail with reference to FIGS. 2-6.

FIG. 2 shows a schematic flowchart of a CEUS imaging method 200 according to an embodiment of the present disclosure. As shown in FIG. 2, the CEUS imaging method 200 may include the following steps:

Step S210: controlling an ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data.

The volumetric data mentioned herein may be data (which may be 3D data or 4D data) obtained by scanning through an ultrasonic volume probe. The ultrasonic volume probe may be either a convex array probe or an area array probe, which is not limited here.

In an embodiment of the present disclosure, by controlling the ultrasonic probe to transmit the ultrasonic waves to the target tissue containing contrast agent, both the volumetric contrast data (also referred to as contrast volumetric data) and volumetric tissue data (also referred to as tissue volumetric data) of the target tissue may be acquired based on the echoes of ultrasonic waves. Here, simultaneous acquisition of the volumetric contrast data and the volumetric tissue data of the target tissue does not necessarily mean that the volumetric contrast data and the volumetric tissue data of the target tissue are acquired at the same time; instead, it may mean that both the volumetric contrast data and the volumetric tissue data can be obtained from the echoes of the ultrasonic waves.

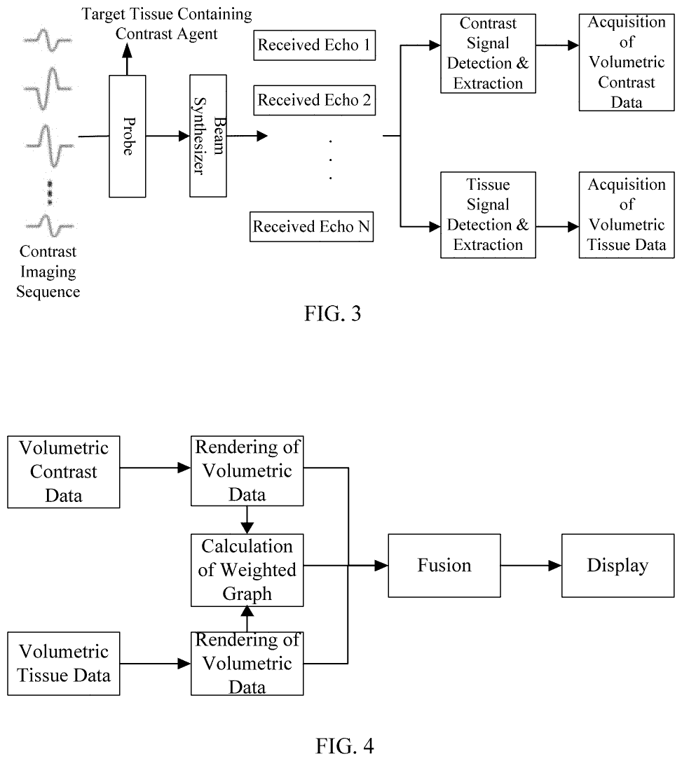

An exemplary acquisition of the volumetric contrast data and the volumetric tissue data in the CEUS imaging method according to an embodiment of the present disclosure is described with reference to FIG. 3. FIG. 3 shows a schematic flowchart of the acquisition of the volumetric contrast data and the volumetric tissue data in the CEUS imaging method according to an embodiment of the present disclosure. As shown in FIG. 3, with regard to the target tissue containing a contrast agent, the acquisition of the volumetric data may be carried out by using an ultrasonic volume (or array) transducer (probe), and the two volumetric data, i.e. the volumetric contrast data and the volumetric tissue data, can be acquired simultaneously according to different transmission sequences.

In an embodiment of the present disclosure, a contrast imaging sequence may be used as the transmission sequence. For example, the contrast imaging sequence used may include two or more transmission pulses with different amplitudes and phases. A relative low transmission voltage may be often used when the transducer is excited by the contrast imaging sequence to prevent the destruction of contrast agent micro-bubbles and realize real-time CEUS imaging. The transducer may successively transmit ultrasonic pulses to the target tissue containing a contrast agent, and successively receive reflected echoes to be inputted into a receiving circuit (such as a beam synthesizer, etc.), to generate a corresponding received echo sequence (for example, received echo 1, received echo 2, . . . , received echo N, where N is a natural number). Then, tissue signals and contrast signals may be detected and extracted according to a corresponding signal detecting and processing mode to generate and store corresponding image data, i.e., acquiring the volumetric contrast data and volumetric tissue data at the same time.

In the embodiment of the present disclosure, the volumetric contrast data obtained in step S210 is referred to as the first contrast data to distinguish it from the second contrast data described below without any other restrictive meaning, and the relationship therebetween is described below. Similarly, in the embodiment of the present disclosure, the volumetric tissue data obtained in step S210 is referred to as the first tissue data to distinguish it from the second tissue data described below without any other restrictive meaning, and the relationship therebetween is described below.

Now, referring to FIG. 2, based on the obtained volumetric contrast data and volumetric tissue data, it is possible to achieve hybrid imaging of the volumetric contrast data and the volumetric tissue data, as described in the following steps.

Step S220: rendering a second contrast data and a second tissue data in real time to acquire a hybrid rendered image of the second contrast data and the second tissue data, the second contrast data containing all or part data of the first contrast data, and the second tissue data containing all or part data of the first tissue data.

In an embodiment of the present disclosure, with regard to the first contrast data and the first tissue data acquired in step S210, fusing and rendering may be performed based on all data of each of them in step S220 (i.e., rendering the first contrast data and the first tissue data in real time to acquire a hybrid rendered image of the first contrast data and the first tissue data, and displaying the hybrid rendered image in step S230 described below), or fusing and rendering may be performed based on part data of both of them or based on part data of one of them and all data of the other to obtain the hybrid rendered image. The part data of either the first contrast data or the first tissue data may include data corresponding to a region of interest (ROI). In order to make the description clearer and more concise, the data rendered in real time in step S220 may be referred to as the second contrast data and the second tissue data, wherein the second contrast data includes all or part of the first contrast data, and the second tissue data includes all or part of the first tissue data.

In an embodiment of the disclosure, the part data mentioned above may include data corresponding to a ROI. The second contrast data may include data of a ROI of the first contrast data; and based on this, the data corresponding to the ROI extracted from the first contrast data is taken as the second contrast data. Similarly, the second tissue data may include data of a ROI of the first tissue data; and in this respect, the data corresponding to the ROI extracted from the first tissue data is taken as the second tissue data.

In an embodiment of the present disclosure, whether for the first contrast data or for the first tissue data, the acquisition of data for respective regions of interest may include but not limit to any one of the following items (1) to (7) or any combination thereof:

(1) Constructing a solid model and setting a ROI by adjusting the size of the solid model to acquire tissue(s) in the ROI, further acquiring tissue data or contrast data within the ROI. The solid model may be in various shapes, such as cuboid, ellipsoid, paraboloid or any shape with a smooth surface, or a combination thereof.

(2) Removing disinterested tissue(s) by means of clipping, erasing, etc. so as to acquire the tissue data or the contrast data within the ROI.

(3) Interactively segmenting tissue(s) of the ROI. For example, the tissue(s) of the ROI may be semi-automatically segmented by using intelligent scissors based on LiveWire algorithm, image segmentation algorithm (such as GrabCut), further acquiring the tissue data or the contrast data within the ROI.

(4) Acquiring a ROI by means of methods like sliding window, thereby obtaining the tissue data or the contrast data corresponding to the ROI. For example, feature extraction methods such as principal component analysis (PCA), linear discriminant analysis (LDA), Harr feature, texture feature, or methods like deep neural network may be used to extract feature(s) within a slide window, then the extracted feature(s) may be matched with a database, and then a discriminator such as K-nearest neighbor (KNN), a support vector machine (SVM), random forest, neural network may be used to classify to determine whether the current slide window is the ROI.

(5) Detecting and recognizing a ROI by using a method of bounding box based on deep learning, thereby obtaining the tissue data or the contrast data within the ROI. For example, a constructed database may be performed with feature learning and parametric regression by stacking a basic convolution layer and a full connection layer. For an input image, the bounding box of a corresponding ROI may be regressed and acquired directly via a network and the category of tissue structure in the ROI may be acquired at the same time, wherein the method adopted here may be region convolutional neural networks (R-CNN), fast R-CNN, Faster-RCNN, single shot multibox detector (SSD), You Only Look Once (YOLO), etc., by which the tissue within the ROI may be acquired automatically.

(6) Detecting and recognizing a ROI by an end-to-end semantic segmentation network method based on deep learning, thereby obtaining the tissue data or the contrast data within the ROI. Such method is similar to the structure of the deep learning-based bounding box mentioned above, except the removal of the full connection layer and the adding of up-sampling or a deconvolution layer which make the sizes of input and output the same, thereby directly obtaining the ROI of the input image and a corresponding category thereof. The method here may be full convolutional networks (FCN), U-Net, mask R-CNN, etc., by which the tissue within the ROI may be acquired automatically.

(7) Locating a target by any of the items (2), (3), (4), (5) or (6) mentioned above and then classifying the target with an additional classifier based on the located result. For example, feature extraction methods (such as PCA, LDA, Harr feature, texture feature, etc.) or deep neural network may be used firstly on a ROI or mask of the target for feature extraction, then the extracted feature(s) may be matched with a database and classified by a discriminator such as KNN, SVM, random forest, neural network or the like to determine whether the current slide window is the ROI. The tissue in the ROI may be acquired automatically, and then the tissue data or contrast data in the ROI may be acquired.

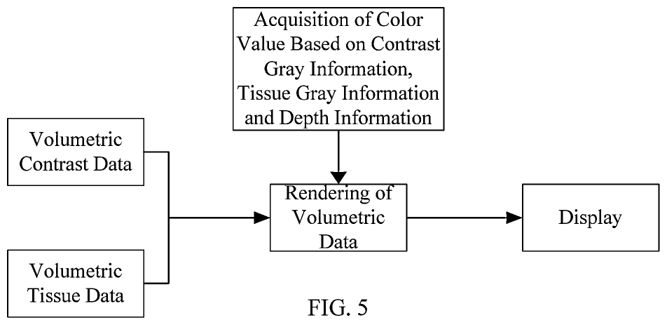

After obtaining the second contrast data and the second tissue data respectively in accordance with the first contrast data and the first tissue data, the second contrast data and the second tissue data may be fused and rendered so as to acquire the hybrid rendered image. In an embodiment of the present disclosure, rendering the second contrast data and the second tissue data to acquire the hybrid rendered image of the second contrast and the second tissue data may further comprise: rendering the second contrast data and the second tissue data respectively in real time, and fusing the rendered results obtained therefrom to acquire the hybrid rendered image; or rendering the second contrast data and the second tissue data simultaneously in real time to acquire the hybrid rendered image. That is, in the present disclosure, the fusion and rendering of the volumetric contrast data and the volumetric tissue data may include that both of the two kinds of data may be rendered separately and then fused and displayed, or be rendered together and then displayed together. Such two fusing and rendering modes are described below with reference to FIG. 4 and FIG. 5, respectively.

FIG. 4 shows a schematic flowchart of an example of fusing and rendering the volumetric contrast data and the volumetric tissue data in the CEUS imaging method according to an embodiment of the present disclosure. As shown in FIG. 4, the volumetric contrast data (i.e. the second contrast data) and the volumetric tissue data (i.e. the first contrast data) are rendered in real time separately, and each rendered result therefrom is calculated for a weighted graph which is used as the basis for the fusion of the two rendered results. Finally, the two rendered results may be fused based on the weighted graph to acquire the hybrid rendered image which may be displayed to users.

Specifically, rendering the second contrast data and the second tissue data in real time respectively and fusing the rendered results obtained therefrom to acquire the hybrid rendered image may further comprise: rendering the second contrast data in real time to obtain a first 3D-rendered image (which may be a 2D image with 3D display effect) and acquiring a color value and a spatial depth value of each pixel in the first 3D-rendered image; rendering the second tissue data in real time to obtain a second 3D-rendered image (which may be a 2D image with 3D display effect) and acquiring a color value and a spatial depth value of each pixel in the second 3D-rendered image; determining a weight of each pixel in the first 3D-rendered image and a weight of each pixel at a corresponding position in the second 3D-rendered image when fusing the color values based on the spatial depth value of each pixel in the first 3D-rendered image and the spatial depth value of each pixel at the corresponding position in the second 3D-rendered image; and calculating a color value of each pixel in a third 3D-rendered image based on the weight of each pixel in the first 3D-rendered image and the weight of each pixel at the corresponding position in the second 3D-rendered image, and mapping the calculated color values to the third 3D-rendered image to acquire the hybrid rendered image. The above process is described in detail below.

In an embodiment of the present disclosure, a rendering mode for real-time rendering of the second contrast data may be surface rendering or volume rendering; similarly, a rendering mode for real-time rendering of the second tissue data may be surface rendering or volume rendering.

There are two main methods for surface rendering, that is, “Delaunay-based” and “MarchingCube extraction from voxel”. Taking MarchingCube as an example, information about iso-surface (i.e. surface contour) of tissue/organ from the volumetric data—the normal vector and vertex coordinates of a triangular surface may be extracted to establish a triangular mesh model, and then volume rendering may be performed in combination with a lighting model, such that a volume render (VR) image can be obtained; wherein the lighting model may include ambient light, scattered light, highlights and so on, and different light source parameters (type, orientation, location, angle) may affect the lighting model to a greater or lesser extent.

Volume rendering mainly adopt a ray-tracing algorithm, and may include the following modes: surface imaging mode for displaying surface information about an object (Surface for short), maximum echo mode for displaying maximum information about the inner of an object (Max for short), minimum echo mode for displaying minimum information about the inner of an object (Min for short), X-ray mode for displaying structure information about the inner of an object (X-Ray for short), shadow imaging mode for displaying surface information of an object based on a global illumination model (Volume Rendering with Global Illumination for short), silhouette mode for displaying internal and external outline information of an object via a translucent effect (Silhouette for short), and time pseudo-color imaging mode for highlighting new contrast data or tissue data about the surface of an object at different moments (wherein the new contrast data or tissue data may be attached with different pseudo-colors with time changes). An appropriate volume rendering mode can be selected based on specific requirements and/or user settings.

Two examples of acquiring a rendered image based on volume rendering are described below.

In one example, multiple rays passing through the contrast (tissue) volumetric data may be emitted based on gaze direction, each ray being progressive according to a fixed step size, and the contrast (tissue) volumetric data along the ray path may be sampled. The opacity of each sampling point may be determined according to the gray value of each sampling point, a cumulative opacity may be acquired by accumulating the opacity of each sampling point on each ray path, and finally the cumulative opacity on each ray path may be mapped to a color value based on a cumulative opacity—color mapping table, said color value may then be mapped to a pixel of a 2D image. In this way, the color value of each pixel at each ray path, further to all ray path, can be acquired to obtain a VR image.

In another example, multiple rays passing through the contrast (tissue) volumetric data may be emitted based on gaze direction, each ray being progressive according to a fixed step size, and the contrast (tissue) volumetric data along the ray path may be sampled. The opacity of each sampling point may be determined according to the gray value of each sampling point, and the opacity of each sampling point may be mapped to a color value through an opacity—color mapping table. Then a cumulative color value may be acquired by accumulating the color value of each sampling point on each ray path, and the cumulative color value may be mapped to a pixel of a 2D image. In this way, the color value of each pixel at each ray path, further to all ray path, can be acquired to obtain a VR image.

The above examples show the way of rendering the second contrast data and the second tissue data separately in real time. A rendered image obtained by real-time rendering of the second contrast data is referred to as a first 3D-rendered image, and a rendered image obtained by real-time rendering of the second tissue data is referred to as a second 3D-rendered image for distinguishing them from each other. When fusing and displaying the first 3D-rendered image and the second 3D-rendered image, a first weighted graph may be determined firstly and then a second weighted graph may be determined based on the first weighted graph, or the second weighted graph may be determined firstly and then the first weighted graph may be determined based on the second weighted graph. The first weighted graph may be a graph of the same size as the first 3D-rendered image, in which the value of each point in the graph (generally ranging from 0 to 1) may represent a weight value that shall be adopted for the color value of each pixel in the first 3D-rendered image when fusing and displaying the first 3D-rendered image and the second 3D-rendered image. Similarly, the second weighted graph may be a graph of the same size as the second 3D-rendered image, in which the value of each point in the graph (generally ranging from 0 to 1) may represent a weight value that shall be adopted for the color value of each pixel in the second 3D-rendered image when fusing and displaying the first 3D-rendered image and the second 3D-rendered image. It can be understood that, taking the weight value in an interval [0, 1] as an example, the sum of the value of any point in the first weighted graph and the value of a corresponding point in the second weighted graph should be equal to 1. The weight value in the interval [0, 1] is only used as an example; and the interval of the weight value is not limited in the present disclosure. Therefore, if the first weighted graph is represented as Map, the second weighted graph is represented as 1-Map; similarly, if the first weighted graph is represented as weight, the second weighted graph is represented as 1-weight. Due to the different principles of surface rendering and volume rendering, the weighted graph adopted in fusion and display is slightly different. Followed is an example of first determining the first weighted graph. Since the first weighted graph refers to the weight values that should be adopted for various pixels of the first 3D-rendered image in fusion and display, the first 3D-rendered image obtained by surface rendering and that by volume rendering are respectively described below.

With regard to the first 3D-rendered image obtained by surface rendering (where the second 3D-rendered image is obtained by surface rendering or volume rendering), the spatial depth value of each pixel in the first 3D-rendered image and that in the second 3D-rendered image may be obtained (wherein, information about the spatial depth may be acquired by obtaining vertex coordinates of a triangular surface for surface rendering; and information about the spatial depth may be acquired by obtaining a starting position where a tissue/organ is sampled for the first time on a ray path and a cutoff position where the ray stops stepping for volume rendering) to calculate the first weighted graph. Since the first weighted graph is calculated based on the spatial depth value of each pixel in the first 3D-rendered image and that in the second 3D-rendered image, the first weighted graph may be referred to as a first spatial-position weighted graph, and the second weighted graph may be referred to as a second spatial-position weighted graph. If the first spatial-position weighted graph is represented as Map, the second spatial-position weighted graph may be represented as 1-Map. The determination of the first spatial-position weighted graph Map and the fusion and display of the first and second 3D-rendered images based thereon are described below.

In an embodiment of the present disclosure, according to the spatial depth value of each pixel in the first 3D-rendered image and that in the second 3D-rendered image, a spatial position relationship between data of pixels in the first 3D-rendered image and data of pixels at corresponding locations in the second 3D-rendered image may be determined, thereby determining the first weighted graph. When determining the spatial position relationship between the data of pixels in the first 3D-rendered image and the data of pixels at corresponding locations in the second 3D-rendered image, an effective spatial depth value interval may be determined for comparison with the spatial depth values of pixels in the second 3D-rendered image by taking the spatial depth values of pixels in the first 3D-rendered image as a reference standard, and the spatial position relationship between the data of pixels in the first 3D-rendered image and the data of pixels at corresponding locations in the second 3D-rendered image may be determined based on the comparison result. Alternatively, an effective spatial depth value interval may be determined for comparison with the spatial depth values of pixels in the first 3D-rendered image by taking the spatial depth values of pixels in the second 3D-rendered image as a reference standard, and the spatial position relationship between the data of pixels in the first 3D-rendered image and the data of pixels at corresponding locations in the second 3D-rendered image may be determined based on the comparison result. The spatial depth value of each pixel in the first 3D-rendered image and that in the second 3D-rendered image may include one or more spatial depth ranges; that is, the spatial depth value of each pixel in the first 3D-rendered image and that in the second 3D-rendered image may each include a minimum and a maximum (in which the minimum and the maximum may be the minimal value and the maximal value of the effective depth range of each pixel, for example, the minimal value and the maximal value of the effective depth range selected by a set gray threshold during volume rendering). Thus the minimum and the maximum of the spatial depth value of each pixel in the first 3D-rendered image and those in the second 3D-rendered image may be acquired for pixel-by-pixel comparison.

The spatial depth value of each pixel in the second 3D-rendered image as the reference standard is taken as an example to describe as follows: with regard to a pixel at any position in the first and second 3D-rendered images, assuming that the minimum and maximum of the spatial depth value of the pixel at the position in the second 3D-rendered image are Y1, Y2 respectively, and that the minimum and maximum of the spatial depth value of the pixel at the position in the first 3D-rendered image are X1, X2 respectively, in the case of X1 being less than or equal to Y1, it may mean that the contrast volumetric data at this position is in front of the tissue volumetric data from users' perspective, and at this point, the value at this position in the first spatial-position weighted graph Map may be set to 1, that is, only the contrast signals are displayed at this position; in the case of X2 being greater than or equal to Y2, it may mean that the contrast volumetric data at this position is behind the tissue volumetric data from user's perspective, and at this point, the value at this position in the first spatial-position weighted graph Map may be set to 0, that is, only the tissue signals are displayed at this position; and in the case of X1 being greater than Y1 and X2 being less than Y2, it may mean that the contrast volumetric data at this position is inside the tissue from users' perspective, and at this point, the value at this position in the first spatial-position weighted graph Map may be set to a value between 0 and 1, that is, the contrast signals and the tissue signals may be displayed at this position in a certain proportion which may be configured according to user requirements or other preset requirements. In this way, the weight of each pixel in the first 3D-rendered image and that in the second 3D-rendered image may be set so as to obtain the first spatial-position weighted graph Map. The above takes the spatial depth value of each pixel in the second 3D-rendered image as the reference standard for illustration, which is not limited herein, for example, the spatial depth value of each pixel in the first 3D-rendered image may be taken as the reference standard. In addition, the sum of the weight values above is 1 for example, which is also unlimited herein.

Based on the determined first spatial-position weighted graph Map mentioned above, the fusion and display of the first and second 3D-rendered images may be carried out. The color value of each pixel of the third 3D-rendered image (i.e. the hybrid rendered image) obtained after the fusion of the first and second 3D-rendered images may be calculated by the following formula (fusion mode):

Color_{Total}=Color_{C}·Map+Color_{B}·(1−Map)

where Color_{Total} represents the color value after fusion, Color_{C} represents the color value of each pixel in the first 3D-rendered image (the contrast image), Color_{B} represents the color value of each pixel in the second 3D-rendered image (the tissue image), and Map represents the first spatial-position weighted graph.

With regard to the first 3D-rendered image obtained by volume rendering (where the second 3D-rendered image may be obtained by surface rendering or volume rendering), the spatial depth value of each pixel in the first 3D-rendered image and that in the second 3D-rendered image together with the cumulative opacity value of each pixel in the first 3D-rendered image may be acquired to calculate the first weighted graph. Since the first weighted graph is calculated based on the spatial depth value of each pixel in the first and second 3D-rendered images and the cumulative opacity of each pixel in the first 3D-rendered image, the first weighted graph may be referred to as a first spatial-position weighted graph, the first weighted graph may be represented as weight herein, the second weighted graph may be represented as 1-weight, and the value of each point in the first weighted graph weight is equal to the value of each point in the first spatial-position weighted graph multiplied by the cumulative opacity value of the pixel corresponding to said point in the first 3D-rendered image, namely weight=Map*Opacity.

Based on the first weighted graph weight, the fusion and display of the first and second 3D-rendered images may be carried out. The calculation formula (fusion mode) for the color value of each pixel of the third 3D rendered image (i.e. the hybrid rendered image) obtained after the fusion of the first and second 3D-rendered images may be expressed as:

Color_{Total}=Color_{C}·weight+Color_{B}·(1−weight)

weight=Map·Opacity_{C}

where Color_{Total} represents the color value after fusion, Color_{C} represents the color value of each pixel in the first 3D-rendered image (the contrast image), Color_{B} represents the color value of each pixel in the second 3D-rendered image (the tissue image), weight represents the first weighted graph, Map represents the first spatial-position weighted graph, and Opacity_{C} represents the cumulative opacity value of each pixel in the first 3D-rendered image. In the case that the first 3D-rendered image is obtained by volume rendering, when the first 3D-rendered image and the second 3D-rendered image are fused and displayed, the cumulative opacity of each pixel in the first 3D-rendered image is added in addition to the aforementioned spatial-position weight, which can make the effect of image obtained after fusion smoother and the edge transition thereof more natural.

The above example shows an example of fusion and rendering of the volumetric contrast data and the volumetric tissue data (i.e., they may be fused and displayed after being separately rendered) with reference to FIG. 4. Another example of fusion and rendering of the volumetric contrast data and the volumetric tissue data is described below with reference to FIG. 5. FIG. 5 shows a schematic flowchart of another example of fusion and rendering of the volumetric contrast data and the volumetric tissue data in the CEUS imaging method according to an embodiment of the present disclosure. As shown in FIG. 5, volume rendering is carried out simultaneously for both the volumetric contrast data (i.e. the second contrast data mentioned above) and the volumetric tissue data (i.e., the second tissue data mentioned above), and the hybrid rendered image may be obtained by acquiring color values based on the gray information, depth information of the second contrast data and the second tissue data.

Specifically, the rendering of the second contrast data and the second tissue data in real time to acquire the hybrid rendered image may comprise: performing volume rendering on the second contrast data and the second tissue data simultaneously to acquire a spatial depth value and a gray value of each sampling point on each ray path during volume rendering, the gray value of each sampling point comprising a gray value of the second contrast data at the point and/or a gray value of the second tissue data at the point; acquiring a color value of each sampling point based on the spatial depth value and the gray value of each sampling point on each ray path, and determining a cumulative color value on each ray path based on the color values of all sampling points on each ray path; and determining a color value of each pixel in a third 3D-rendered image based on the cumulative color value on each ray path, and mapping the cumulative color value to the third 3D-rendered image to acquire the hybrid rendered image.

The acquisition of the color value of each sampling point based on the spatial depth value and the gray value of each sampling point on each ray path may comprise: according to a predetermined 3D color index table, acquiring the color value of each sampling point based on the spatial depth value and the gray value of each sampling point on each ray path, the 3D color index table containing 3D variables that are a contrast gray value, a tissue gray value and a spatial depth value, the 3D variables corresponding to one color value; or, according to a predetermined mapping function, acquiring the color value of each sampling point based on the spatial depth value and the gray value of each sampling point on each ray path, the predetermined mapping function including three variables, namely, a contrast gray value, a tissue gray value and a spatial depth value, and a function result of the predetermined mapping function being a color value.

In this embodiment, with ray tracing algorithm, multiple rays passing through the contrast volumetric data and the tissue volumetric data may be emitted based on gaze direction, each ray being progressive according to a fixed step size, and the contrast volumetric data and the tissue volumetric data along the ray path may be sampled to acquire the gray value of the contrast volumetric data and/or the gray value of the tissue volumetric data at each sampling point, and then the color value may be obtained by indexing the 3D color index table with information about a step depth of a current ray or by the predetermined mapping function, thereby acquiring the color value of each sampling point. Then, the color value of each sampling point on each ray path is accumulated, and the accumulated color value is mapped to a pixel of a 2D image. In this way, the color value of each pixel at each ray path, further to all ray path, can be obtained to acquire a VR image, thus acquiring a final hybrid rendered image. That is to say, simultaneous rendering of the second contrast data and the second tissue data to obtain the hybrid rendered image may be expressed by the following formula:

$$C o l o {r}_{r a y} = 3 DColorTexture ( {value}_{C} , {value}_{B} , depth ) \\ {Color}_{Total} = {\sum }_{start}^{e n d} C o l o {r}_{r a y}$$

where Color_{ray} represents the color value of a current sampling point, value_{C} represents a contrast gray value of the current sampling point, value_{B} represents a tissue gray value of the current sampling point, depth represents information about ray depth of the current sampling point, 3DColorTexture( ) represents the 3D color index table or the predetermined mapping function, Color_{Total} represents the cumulative color value of each sampling point on the current ray path, start represents the first sampling point on the current ray path, and end represents the last sampling point on the current ray path.

Step S230: displaying the hybrid rendered image in real time.

In an example, the hybrid rendered image may comprise at least part of a rendered image obtained by real-time rendering of the second contrast data and at least part of a rendered image obtained by real-time rendering of the second tissue data.

It should be noted that real-time imaging of fusing the ultrasonic volume contrast and volume tissue, that is collecting volumetric data of both tissue and contrast in real time and displaying the hybrid image of tissue and contrast after being rendered in real time, can be realized according to the present disclosure. Generally speaking, its imaging frame rate is above 0.8 VPS (Volume Per Seconds). Compared with CT, MRI and other non-real-time imaging, the present disclosure can greatly reduce the time consumption of the imaging process.

As mentioned above, the second contrast data and the second tissue data are volumetric data (i.e., 3D or 4D data). One or more frames of hybrid rendered images may thus obtained based on the aforesaid steps S210 to S220. In an embodiment of the present disclosure, after multiple frames of hybrid rendered images are obtained, they may be displayed in a multi-frame dynamic manner, for example, the multi-frame hybrid rendered images may be displayed dynamically in chronological order. In an example, with regard to each frame of the hybrid rendered image, a different image feature (such as a different color) may be used to display the part thereof that represents contrast data or the part that represents tissue data. For example, the part of the hybrid rendered image representing contrast data is shown in yellow, and the part of the hybrid rendered image representing tissue data is shown in gray. In this way, during dynamically displaying the multi-frame hybrid rendered images, real-time changes in the spatial position relationship between the contrast agent and the tissue can be observed.

In an embodiment of the present disclosure, the target tissue mentioned above may include an oviduct region; further, the hybrid rendered image may be performed with feature extraction, and based on the result of feature extraction, an analysis result of the oviduct region may be outputted.

It should be noted that, based on the hybrid rendered image obtained in step S230, the analysis result of the oviduct presented in the hybrid rendered image may be obtained based on the features extracted from the hybrid rendered image to provide a diagnostic basis for the oviduct of the target object. When more than one frame of the hybrid rendered image is obtained, feature extraction may be carried out for each frame of the hybrid rendered image, and respective analysis result of the oviduct region corresponding to each frame of the hybrid rendered image may be outputted. It is possible to output the analysis result of the oviduct region corresponding to one frame of the hybrid rendered image based on the results of feature extraction of the multiple frames of the hybrid rendered images (for example, based on the results of feature extraction of N-frame hybrid rendered images, the analysis result of oviduct region corresponding to the last frame, i.e. the Nth frame, of the hybrid rendered image are outputted, where N is a natural number greater than 1).

In an embodiment of the present disclosure, each frame of the hybrid rendered image may be performed with feature extraction based on image processing algorithm(s), such as principal components analysis (PCA), linear discriminant analysis (LDA), Harr feature, texture feature and so on. In an embodiment of the present disclosure, each frame of the hybrid rendered image may be performed with feature extraction based on neutral network including AlexNet, VGG, ResNet, MobileNet, DenseNet, EfficientNet, EfficientDet.

In an embodiment of the present disclosure, the output of an analysis result of the oviduct region based on the result of feature extraction may comprise: matching the result of feature extraction with feature(s) pre-stored in a database, classifying by a discriminator to output the classified result as the analysis result of the oviduct region. For example, the discriminator may include but not limit to K-nearest neighbor (KNN), support vector machines (SVM), random forest, neural network, etc.

In an embodiment of the present disclosure, the analysis result of the oviduct region may include at least one relevant attribute of the oviduct of the target object. Exemplarily, relevant attribute(s) may include patency, shape, the presence of fluid accumulated in fimbriated extremity, and the presence of cyst. The attribute of patency may include: normal, partially obstructed, completely obstructed, lack, etc.; and the attribute of shape may include: distorted, too long, too short, and so on. In addition, the analysis result of the oviduct region may also include probability of each relevant attribute determined, such as the probability that the oviduct is partially obstructed, or the probability that the oviduct distorted. For example, the probability for each relevant attribute may range from 0 to 100%. As mentioned above, each frame of the hybrid rendered image may be performed with feature extraction and classification to output a corresponding analysis result, that is, at least one of the aforesaid relevant attributes and the probability of each relevant attribute of the oviduct of the target object determined based on one or several frames of the hybrid rendered images.

In a further embodiment of the present disclosure, the analysis result of the oviduct region may also be a score result of the oviduct of the target object, wherein the score result may be determined based on the output of each relevant attribute and the probability of each relevant attribute. In one example, when the attribute of patency may be determined as normal after feature extraction and classification by a discriminator, and the probability thereof may be 100%, the score result thereof may be normal 100. In another example, when the attribute of patency may be determined as completely obstructed after feature extraction and classification by a discriminator, and the probability thereof may be 100%, the score result thereof may be completely obstructed 100. In other examples, a composite score may be determined from respective probabilities of multiple relevant attributes.

In an embodiment of the present disclosure, a corresponding analysis result of the oviduct may be marked on at least one frame of hybrid rendered image, and the marked hybrid rendered image may be displayed to users, for example, displaying a hybrid rendered image of normal oviduct with a marked score result “normal: 100”, or displaying a hybrid rendered image of completely obstructed oviduct with a marked score result “completely obstructed: 100”. In this embodiment, a hybrid rendered image marked with an analysis result of an oviduct may be displayed to a user (e.g. a doctor), from which both a contrast region and a tissue region can be seen in the hybrid rendered image, thereby enabling the user to intuitively understand and observe the spatial position relationship and flow of the contrast agent in the tissue. Also the user can intuitively understand the automatic analysis result of the oviduct of the target object by means of the marked result of the hybrid rendered image. Therefore a reference for the doctor's diagnosis can be provided to further improve the diagnosis efficiency. In another embodiment, it is also possible to display the hybrid rendered image and the analysis result of the oviduct separately.

In a further embodiment of the present disclosure, pseudo-color display may be performed in addition to the aforesaid multi-frame dynamic display. Exemplarily, with regard to a newly displayable contrast data in front of the tissue data in a current frame of hybrid rendered image relative to a previous frame of hybrid rendered image, it may be displayed in a color different from the previous one to show the position of the contrast data newly in the tissue data. For example, whereas the part of the hybrid rendered image representing contrast data is shown in yellow in the previous example, in this embodiment the part representing additional contrast data could be shown in a color different from yellow, such as blue. In this way, during displaying the multiple frames of the hybrid rendered images in a dynamic manner, real-time changes in the spatial position relationship between the contrast agent and the tissue can be observed, so does the flow of the contrast agent in the tissue.

In a further embodiment of the present disclosure, after acquiring the hybrid rendered image of a current frame, the display thereof may be adjusted based on a received user instruction. For example, if the user expects that all tissue data or all contrast data can be displayed in the current frame of the hybrid rendered image, or that the tissue data and the contrast data can be shown in a desired transparency, the weight in the aforesaid weighted graph for fusing and displaying the current frame may be adjusted based on a user instruction to obtain a display effect expected by the user. In this embodiment, the current frame of the hybrid rendered image can be adjusted by the user, realizing more flexible hybrid imaging of volumetric contrast and tissue data.

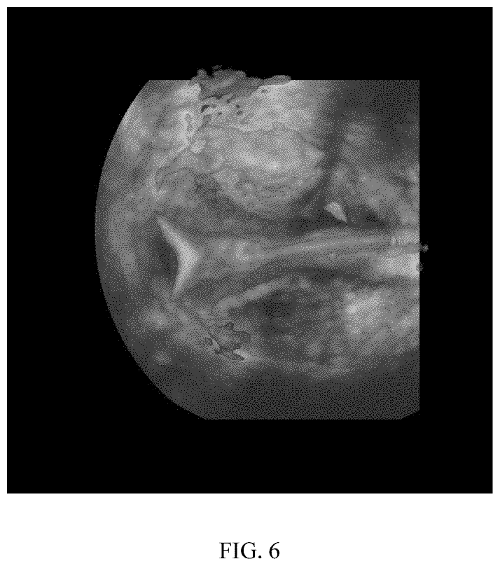

The above example shows fusing and rendering performed on the volumetric contrast data and the volumetric tissue data by the CEUS imaging method according to an embodiment of the present disclosure, and a final acquired hybrid rendered image of the volumetric contrast data and the volumetric tissue data may be shown in FIG. 6. FIG. 6 shows an exemplary schematic diagram of a hybrid rendered image resulted from the CEUS imaging method according to an embodiment of the present disclosure. As shown in FIG. 6, both the contrast region and the tissue region can be seen in the hybrid rendered image, enabling the user to intuitively understand and observe the spatial position relationship so as to acquire more clinical information.

Based on the above description, the volumetric contrast data and the volumetric tissue data can be collected simultaneously and then fused and rendered to acquire a hybrid rendered image according to the CEUS imaging method in an embodiment of the present disclosure, helping users more intuitively understand and observe the real-time spatial position relationship of a contrast agent in tissues, further acquiring more clinical information.

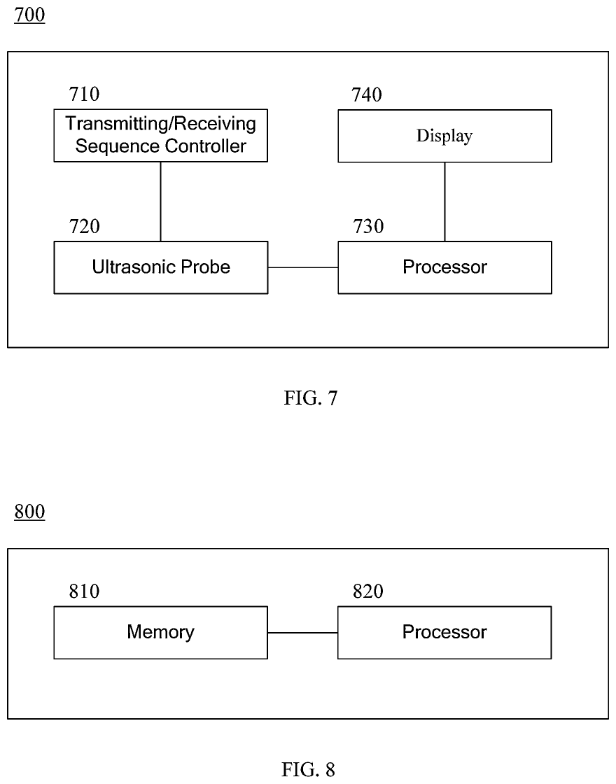

An ultrasound imaging apparatus provided according to another aspect of the present disclosure is described as follows with reference to FIG. 7 and FIG. 8. FIG. 7 shows a schematic block diagram of an ultrasound imaging apparatus 700 according to an embodiment of the present disclosure. As shown in FIG. 7, the ultrasound imaging apparatus may include a transmitting/receiving sequence controller 710, an ultrasonic probe 720, a processor 730 and a display 740. The transmitting/receiving sequence controller 710 may be used to control the ultrasonic probe 720 to transmit ultrasonic waves to a target tissue containing contrast agent and receive echoes of the ultrasonic waves to acquire a first contrast data and a first tissue data in real time based on the echoes of the ultrasonic waves. The first contrast data and the first tissue data may be both volumetric data. The processor 730 may be used to perform real-time rendering on a second contrast data and a second tissue data to obtain a hybrid rendered image of the second contrast data and the second tissue data. The second contrast data may include all or part of the first contrast data, and the second tissue data may include all or part of the first tissue data. The display 740 may be used to display the hybrid rendered image in real time.

In an embodiment of the present disclosure, the part data may contain data corresponding to a ROI, and the processor 730 may be further configured to extract the data corresponding to a ROI from the first contrast data as the second contrast data; and/or to extract the data corresponding to a ROI from the first tissue data as the second tissue data.

In an embodiment of the present disclosure, the real-time rendering of the second contrast data and the second tissue data performed by the processor 730 to acquire the hybrid rendered image of the second contrast data and the second tissue data may include: rendering the second contrast data and the second tissue data separately in real time, and fusing rendered results obtained therefrom to acquire the hybrid rendered image; or rendering the second contrast data and the second tissue data simultaneously in real time to acquire the hybrid rendered image.

In an embodiment of the present disclosure, the real-time rendering of the second contrast data and the second tissue data and the fusion of rendered results obtained therefrom to acquire the hybrid rendered image, which are performed by the processor 730, may include: rendering the second contrast data in real time to obtain a first 3D-rendered image and acquiring a color value and a spatial depth value of each pixel in the first 3D-rendered image; rendering the second tissue data in real time to obtain a second 3D-rendered image and acquiring a color value and a spatial depth value of each pixel in the second 3D-rendered image; determining a weight of each pixel in the first 3D-rendered image and a weight of each pixel at a corresponding position in the second 3D-rendered image when fusing the color values based on the spatial depth value of each pixel in the first 3D-rendered image and the spatial depth value of each pixel at the corresponding position in the second 3D-rendered image; and calculating a color value of each pixel in a third 3D-rendered image based on the weight of each pixel in the first 3D-rendered image and the weight of each pixel at the corresponding position in the second 3D-rendered image, and mapping the calculated color values to the third 3D-rendered image to acquire the hybrid rendered image.

In an embodiment of the present disclosure, a rendering mode for real-time rendering of both the second contrast data and the second tissue data by the processor 730 may be surface rendering.

In an embodiment of the present disclosure, a rendering mode for real-time rendering of the second contrast data and/or the second tissue data used by the processor 730 may be volume rendering, and the processor 730 may determine a weight of each pixel in the first 3D-rendered image and a weight of each pixel at a corresponding position in the second 3D-rendered image when fusing the color values also based on a cumulative opacity value of each pixel in the first 3D-rendered image and/or a cumulative opacity value of each pixel at the corresponding position in the second 3D-rendered image.

In an embodiment of the present disclosure, the simultaneous real-time rendering of the second contrast data and the second tissue data performed by the processor 730 to acquire the hybrid rendered image may include: performing volume rendering on the second contrast data and the second tissue data simultaneously to acquire a spatial depth value and a gray value of each sampling point on each ray path during volume rendering, the gray value of each sampling point comprising a gray value of the second contrast data at the point and/or a gray value of the second tissue data at the point; acquiring a color value of each sampling point based on the spatial depth value and the gray value of each sampling point on each ray path, and determining a cumulative color value on each ray path based on the color values of all sampling points on each ray path; and determining a color value of each pixel in a third 3D-rendered image based on the cumulative color value on each ray path, and mapping the cumulative color value to the third 3D-rendered image to acquire the hybrid rendered image.

In an embodiment of the present disclosure, the acquisition of a color value of each sampling point based on the spatial depth value and the gray value of each sampling point on each ray path performed by the processor 730 may include: according to a predetermined 3D color index table, acquiring the color value of each sampling point based on the spatial depth value and the gray value of each sampling point on each ray path, the 3D color index table containing 3D variables that are a contrast gray value, a tissue gray value and a spatial depth value, the 3D variables corresponding to one color value; or, according to a predetermined mapping function, acquiring the color value of each sampling point based on the spatial depth value and the gray value of each sampling point on each ray path, the predetermined mapping function including three variables, namely, a contrast gray value, a tissue gray value and a spatial depth value, and a function result of the predetermined mapping function being a color value.

In an embodiment of the present disclosure, the extraction of data corresponding to a ROI performed by the processor 730 may be realized based on a deep learning device.

In an embodiment of the present disclosure, the acquisition of the first contrast data and the first tissue data based on the echoes of the ultrasonic waves performed by the ultrasonic probe 720 may include: acquiring a first contrast signal and a first tissue signal based on the echoes of the ultrasonic waves; and acquiring the first contrast data in real time based on the first contrast signal and acquiring the first tissue data in real time based on the first tissue signal.

In general, the ultrasound imaging apparatus 700 according to an embodiment of the present disclosure may be used to perform the CEUS imaging method 200 described above according to an embodiment of the present disclosure. Those skilled in the art may understand the structure and operation of the ultrasound imaging apparatus 700 based on the description above. For the sake of brevity, some of the details above are not repeated here.

Based on the above description, the volumetric contrast data and the volumetric tissue data can be collected simultaneously and then fused and rendered to acquire a hybrid rendered image according to the ultrasound imaging apparatus in an embodiment of the present disclosure, helping users more intuitively understand and observe the real-time spatial position relationship of a contrast agent in tissues, further acquiring more clinical information.

FIG. 8 shows a schematic block diagram of an ultrasound imaging apparatus 800 according to an embodiment of the present disclosure. The ultrasound imaging apparatus 800 may comprise a memory 810 and a processor 820.

The memory 810 may store program(s) configured to implement corresponding step(s) in CEUS imaging method 200 according to an embodiment of the present disclosure. The processor 820 may be configured to run the program stored in memory 810 to perform the corresponding steps of CEUS imaging method 200 according to an embodiment of the present disclosure.

A CEUS imaging method may also be provided in accordance with yet another aspect of the present disclosure. The method may include: controlling an ultrasonic probe to transmit ultrasonic waves to a target tissue containing a contrast agent and receive echoes of the ultrasonic waves to acquire a first contrast data and a first tissue data in real time based on the echoes of the ultrasonic waves, the first contrast data and the first tissue data being volumetric data; rendering the first contrast data in real time to obtain a first 3D-rendered image, and rendering the first tissue data in real time to obtain a second 3D-rendered image; and simultaneously displaying the first 3D-rendered image and the second 3D-rendered image. In this embodiment, the volumetric contrast data and the volumetric tissue data may be acquired from the echoes of the ultrasonic waves, and they may be rendered in real time separately to obtain respective hybrid rendered images that may be displayed simultaneously on the same interface, helping users to observe the real-time spatial position relationship of a contrast agent in tissues, further acquiring more clinical information.

An ultrasound imaging apparatus that may be used to implement the aforesaid CEUS imaging method may also be provided in accordance with still yet another aspect of the present disclosure. Specifically, the ultrasound imaging apparatus may include an ultrasonic probe, a transmitting/receiving sequence controller, a processor and a display, wherein the transmitting/receiving sequence controller may be configured to control the ultrasonic probe to transmit ultrasonic waves to a target tissue containing a contrast agent and receive echoes of the ultrasonic waves to acquire a first contrast data and a first tissue data in real time based on the echoes of the ultrasonic waves, the first contrast data and the first tissue data being volumetric data; the processor may be configured to render the first contrast data in real time to obtain a first 3D-rendered image, and render the first tissue data in real time to obtain a second 3D-rendered image; and the display may be configured to simultaneously display the first 3D-rendered image and the second 3D-rendered image in real time. Those skilled in the art may understand the structure and operation of the ultrasound imaging apparatus based on the description above. For the sake of brevity, some of the details above are not repeated here.

In addition, according to an embodiment of the present disclosure, a storage medium on which program instruction(s) may be stored is provided to perform the corresponding step(s) of the CEUS imaging method of an embodiment of the present disclosure when the program instruction(s) may be run by a computer or a processor. The storage medium may include, for example, a memory card of a smart phone, a memory component of a tablet computer, a hard disk of a personal computer, a read-only memory (ROM), erasable programmable read-only memory (EPROM), portable compact disk read-only memory (CD-ROM), USB memory, or any combination of the above storage media. A computer readable storage medium may be any combination of one or more computer readable storage media.

In addition, according to an embodiment of the present disclosure, a computer program is provided which can be stored in the cloud or on a local storage medium. The corresponding steps of the CEUS imaging method of an embodiment of the present disclosure may be performed when the computer program is run by a computer or a processor.

Based on the above description, with the CEUS imaging methods, the ultrasound imaging apparatus and the storage media according to embodiments of the present disclosure, volumetric contrast data and volumetric tissue data can be collected simultaneously and then fused and rendered to acquire a hybrid rendered image, helping users more intuitively understand and observe the real-time spatial position relationship of a contrast agent in tissues, and further acquire more clinical information.

While exemplary embodiments have been described herein with reference to the accompanying drawings, it should be understood that the above example embodiments are merely illustrative and are not intended to limit the scope of the disclosure thereto. Those skilled in the art may make various changes and modifications therein without departing from the scope and spirit of the disclosure. All such changes and modifications are intended to be included in the scope of the disclosure as claimed in the appended claims.

A person of ordinary skill in the art may be aware that, in combination with the examples described in the embodiments disclosed in this specification, units and algorithm steps may be implemented by using electronic hardware or a combination of computer software and electronic hardware. Whether the functions are performed by hardware or software depends on particular applications and design constraint conditions of the technical solutions. Those skilled in the art could use different methods to implement the described functions for each particular application, but such implementation should not be considered to be beyond the scope of the disclosure.

In several embodiments provided in the present disclosure, it should be understood that the disclosed devices and methods may be implemented in other ways. For example, the device embodiments described above are merely exemplary. For example, the division of units is merely a logical function division. In actual implementations, there may be other division methods. For example, a plurality of units or components may be combined or integrated into another device, or some features may be omitted or not implemented.

A large number of specific details are explained in this specification provided herein. However, it can be understood that the embodiments of the disclosure can be practiced without these specific details. In some instances, well-known methods, structures, and technologies are not shown in detail, so as not to obscure the understanding of this description.

Similarly, it should be understood that in order to simplify the disclosure and help to understand one or more of various aspects of the disclosure, in the description of the exemplary embodiments of the disclosure, various features of the disclosure are sometimes together grouped into an individual embodiment, figure or description thereof. However, the method of the disclosure should not be construed as reflecting the following intention, namely, the disclosure set forth requires more features than those explicitly stated in each claim. More precisely, as reflected by the corresponding claims, the inventive point thereof lies in that features that are fewer than all the features of an individual embodiment disclosed may be used to solve the corresponding technical problem. Therefore, the claims in accordance with the particular embodiments are thereby explicitly incorporated into the particular embodiments, wherein each claim itself serves as an individual embodiment of the disclosure.

Those skilled in the art should understand that, in addition to the case where features are mutually exclusive, any combination may be used to combine all the features disclosed in this specification (along with the appended claims, abstract, and drawings) and all the processes or units of any of methods or devices as disclosed. Unless explicitly stated otherwise, each feature disclosed in this specification (along with the appended claims, abstract, and drawings) may be replaced by an alternative feature that provides the same, equivalent, or similar object.

Furthermore, those skilled in the art should understand that although some of the embodiments described herein comprise some but not other features included in other embodiments, combinations of features of different embodiments are meant to be within the scope of the disclosure, and form different embodiments. For example, in the claims, any one of the embodiments set forth thereby can be used in any combination.

Various embodiments regarding components in the disclosure may be implemented in hardware, or implemented by software modules running on one or more processors, or implemented in a combination thereof. It should be understood for those skilled in the art that a microprocessor or a digital signal processor (DSP) may be used in practice to implement some or all of the functions of some modules according to the embodiments of the disclosure. The disclosure may further be implemented as an apparatus program (e.g. a computer program and a computer program product) for executing some or all of the methods described herein. Such a program for implementing the disclosure may be stored on a computer-readable medium, or may be in the form of one or more signals. Such a signal may be downloaded from an Internet website, or provided on a carrier signal, or provided in any other form.

It should be noted that the description of the disclosure made in the above-mentioned embodiments is not to limit the disclosure, and those skilled in the art may design alternative embodiments without departing from the scope of the appended claims. In the claims, any reference signs placed between parentheses should not be construed as limitation on the claims. The word “comprising” does not exclude the presence of elements or steps not listed in a claim. The word “a” or “an” preceding an element does not exclude the presence of a plurality of such elements. The disclosure may be implemented by means of hardware comprising several different elements and by means of an appropriately programmed computer. In unit claims listing several devices, several of these devices may be specifically embodied by one and the same item of hardware. The use of the terms “first”, “second”, “third”, etc. does not indicate any order. These terms may be interpreted as names.

The above is only the specific embodiment of the present disclosure or the description of the specific embodiment, and the protection scope of the present disclosure is not limited thereto. Any changes or substitutions should be included within the protection scope of the present disclosure. The protection scope of the present disclosure shall be subject to the protection scope of the claims.

1. A contrast enhanced ultrasound imaging method, comprising:
controlling an ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data;
rendering a second contrast data and a second tissue data in real time to acquire a hybrid rendered image of the second contrast data and the second tissue data, the second contrast data containing all or part data of the first contrast data, and the second tissue data containing all or part data of the first tissue data; and
displaying the hybrid rendered image in real time.

2. The method according to claim 1, wherein the part data contains data corresponding to a region of interest, and the method further comprises:
extracting the data corresponding to the region of interest from the first contrast data as the second contrast data; and/or extracting the data corresponding to the region of interest from the first tissue data as the second tissue data.

3. The method according to claim 1, wherein said rendering a second contrast data and a second tissue data in real time to acquire a hybrid rendered image of the second contrast data and the second tissue data comprises:
rendering the second contrast data and the second tissue data respectively in real time, and fusing rendered results obtained therefrom to acquire the hybrid rendered image; or
rendering the second contrast data and the second tissue data simultaneously in real time to acquire the hybrid rendered image.

4. The method according to claim 3, wherein said rendering the second contrast data and the second tissue data respectively in real time and fusing rendered results obtained therefrom to acquire the hybrid rendered image comprises:
rendering the second contrast data in real time to obtain a first 3D-rendered image and acquiring a color value and a spatial depth value of each pixel in the first 3D-rendered image;
rendering the second tissue data in real time to obtain a second 3D-rendered image and acquiring a color value and a spatial depth value of each pixel in the second 3D-rendered image;
determining a weight of each pixel in the first 3D-rendered image and a weight of each pixel at a corresponding position in the second 3D-rendered image when fusing the color values based on the spatial depth value of each pixel in the first 3D-rendered image and the spatial depth value of each pixel at the corresponding position in the second 3D-rendered image; and
calculating a color value of each pixel in a third 3D-rendered image based on the weight of each pixel in the first 3D-rendered image and the weight of each pixel at the corresponding position in the second 3D-rendered image, and mapping the calculated color values to the third 3D-rendered image to acquire the hybrid rendered image.

5. The method according to claim 4, wherein a rendering mode for real-time rendering of both the second contrast data and the second tissue data is surface rendering.

6. The method according to claim 4, wherein a rendering mode for real-time rendering of the second contrast data and/or the second tissue data is volume rendering, and
said determining a weight of each pixel in the first 3D-rendered image and a weight of each pixel at a corresponding position in the second 3D-rendered image when fusing the color values is also based on a cumulative opacity value of each pixel in the first 3D-rendered image and/or a cumulative opacity value of each pixel at the corresponding position in the second 3D-rendered image.

7. The method according to claim 3, wherein said rendering a second contrast data and a second tissue data in real time to acquire a hybrid rendered image comprises:
performing volume rendering on the second contrast data and the second tissue data simultaneously to acquire a spatial depth value and a gray value of each sampling point on each ray path during volume rendering, the gray value of each sampling point comprising a gray value of the second contrast data at the point and/or a gray value of the second tissue data at the point;
acquiring a color value of each sampling point based on the spatial depth value and the gray value of each sampling point on each ray path, and determining a cumulative color value on each ray path based on the color values of all sampling points on each ray path; and
determining a color value of each pixel in the third 3D-rendered image based on the cumulative color value on each ray path, and mapping the cumulative color value to the third 3D-rendered image to acquire the hybrid rendered image.

8. The method according to claim 7, wherein said acquiring a color value of each sampling point based on the spatial depth value and the gray value of each sampling point on each ray path comprises:
according to a predetermined 3D color index table, acquiring the color value of each sampling point based on the spatial depth value and the gray value of each sampling point on each ray path, the 3D color index table containing 3D variables that are a contrast gray value, a tissue gray value and a spatial depth value respectively, the 3D variables corresponding to one color value; or,
according to a predetermined mapping function, acquiring the color value of each sampling point based on the spatial depth value and the gray value of each sampling point on each ray path, the predetermined mapping function including three variables, namely, a contrast gray value, a tissue gray value and a spatial depth value, and a function result of the predetermined mapping function being a color value.

9. The method according to claim 1, wherein the hybrid rendered image comprises at least part of a rendered image obtained by real-time rendering of the second contrast data and at least part of a rendered image obtained by real-time rendering of the second tissue data.

10. The method according to claim 1, wherein said acquiring a first contrast data and a first tissue data in real time based on the echo of the ultrasonic wave comprises:
acquiring a first contrast signal and a first tissue signal based on the echo of the ultrasonic wave; and
acquiring the first contrast data in real time based on the first contrast signal, and acquiring the first tissue data in real time based on the first tissue signal.

11. The method according to claim 1, wherein the target tissue comprises an oviduct region, and the method further comprises:
performing feature extraction on the hybrid rendered image, and outputting an analysis result of the oviduct region based on a result of the feature extraction; and
displaying the analysis result.

12. An ultrasound imaging apparatus, comprising an ultrasonic probe, a transmitting/receiving sequence controller, a processor and a display,
the transmitting/receiving sequence controller configured for controlling the ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data;
the processor configured for rendering a second contrast data and a second tissue data in real time to acquire a hybrid rendered image of the second contrast data and the second tissue data, the second contrast data containing all or part data of the first contrast data, and the second tissue data containing all or part data of the first tissue data; and
the display configured for displaying the hybrid rendered image in real time.

13. The apparatus according to claim 12, wherein the part data contains data corresponding to a region of interest, and the processor is further configured for:
extracting the data corresponding to the region of interest from the first contrast data as the second contrast data; and/or extracting the data corresponding to the region of interest from the first tissue data as the second tissue data.

14. The apparatus according to claim 12 or 13, wherein said processor rendering a second contrast data and a second tissue data in real time to acquire a hybrid rendered image of the second contrast data and the second tissue data comprises:
rendering the second contrast data and the second tissue data respectively in real time, and fusing rendered results obtained therefrom to acquire the hybrid rendered image; or
rendering the second contrast data and the second tissue data simultaneously in real time to acquire the hybrid rendered image.

15. The apparatus according to claim 14, wherein said processor rendering the second contrast data and the second tissue data respectively in real time and fusing rendered results obtained therefrom to acquire the hybrid rendered image comprises:
rendering the second contrast data in real time to obtain a first 3D-rendered image and acquiring a color value and a spatial depth value of each pixel in the first 3D-rendered image;
rendering the second tissue data in real time to obtain a second 3D-rendered image and acquiring a color value and a spatial depth value of each pixel in the second 3D-rendered image;
determining a weight of each pixel in the first 3D-rendered image and a weight of each pixel at a corresponding position in the second 3D-rendered image when fusing the color values based on the spatial depth value of each pixel in the first 3D-rendered image and the spatial depth value of each pixel at the corresponding position in the second 3D-rendered image; and
calculating a color value of each pixel in a third 3D-rendered image based on the weight of each pixel in the first 3D-rendered image and the weight of each pixel at the corresponding position in the second 3D-rendered image, and mapping the calculated color values to the third 3D-rendered image to acquire the hybrid rendered image.

16.-17. (canceled)

18. The apparatus according to claim 14, wherein said processor rendering a second contrast data and a second tissue data in real time to acquire a hybrid rendered image comprises:
performing volume rendering on the second contrast data and the second tissue data simultaneously to acquire a spatial depth value and a gray value of each sampling point on each ray path during volume rendering, the gray value of each sampling point comprising a gray value of the second contrast data at the point and/or a gray value of the second tissue data at the point;
acquiring a color value of each sampling point based on the spatial depth value and the gray value of each sampling point on each ray path, and determining a cumulative color value on each ray path based on the color values of all sampling points on each ray path; and
determining a color value of each pixel in a third 3D-rendered image based on the cumulative color value on each ray path, and mapping the cumulative color value to the third 3D-rendered image to acquire the hybrid rendered image.

19. (canceled)

20. The apparatus according to claim 14, wherein the hybrid rendered image comprises at least part of a rendered image obtained by real-time rendering of the second contrast data and at least part of a rendered image obtained by real-time rendering of the second tissue data.

21. The apparatus according to claim 12, wherein said ultrasonic probe acquiring a first contrast data and a first tissue data in real time based on the echo of the ultrasonic wave comprises:
acquiring a first contrast signal and a first tissue signal based on the echo of the ultrasonic wave; and
acquiring the first contrast data in real time based on the first contrast signal, and acquiring the first tissue data in real time based on the first tissue signal.

22. The apparatus according to claim 12, wherein the target tissue comprises an oviduct region,
the processor is further configured for performing feature extraction on the hybrid rendered image, and outputting an analysis result of the oviduct region based on a result of the feature extraction; and
the display is further configured for displaying the analysis result.

23. A contrast enhanced ultrasound imaging method, comprising:
controlling an ultrasonic probe to transmit an ultrasonic wave to a target tissue containing a contrast agent, receive an echo of the ultrasonic wave, and acquire a first contrast data and a first tissue data in real time based on the echo of the ultrasonic wave, the first contrast data and the first tissue data being volumetric data;
rendering the first contrast data and the first tissue data in real time to acquire a hybrid rendered image of the first contrast data and the first tissue data; and
displaying the hybrid rendered image in real time.

24.-27. (canceled)
